(19) United States
(12) Patent Application Publication
(10) Pub. No.: US 2017/0042160 A1
(43) Pub. Date: Feb. 16, 2017

(54) ACIDIC ELECTROLYZED WATER AND MANUFACTURING METHOD THEREFOR, DISINFECTANT AND CLEANSER CONTAINING ACIDIC ELECTROLYZED WATER, DISINFECTING METHOD USING ACIDIC ELECTROLYZED WATER, AND MANUFACTURING DEVICE FOR ACIDIC ELECTROLYZED WATER

(71) Applicant: MOLEX INCORPORATED, Lisle, IL (US)

(72) Inventors: Megumi MURAMOTO, Yamato (JP); Kousuke TAKETOMI, Yamato (JP)

(73) Assignee: Molex, LLC, Lisle, IL (US)

(21) Appl. No.: 15/301,122

(22) PCT Filed: May 1, 2015
(86) PCT No.: PCT/US15/28812

§ 371 Date: Sep. 30, 2016

(30) Foreign Application Priority Data
May 1, 2014 (JP) 2014-094442

(51) Int. Cl. A01N 59/08; A61L 9/015; C02F 1/461

(52) U.S. Cl. CPC A01N 59/08; C02F 1/4618; A61L 9/015; C02F 2001/46185; C02F 2201/46115; C02F 2303/04; C02F 2301/08; A61L 2209/213

(57) ABSTRACT

An acidic electrolyzed water and a manufacturing method therefor, a disinfectant and a cleanser containing acidic electrolyzed water, and a disinfecting method using acidic electrolyzed water which has disinfecting power for a long period of time, and which leaves behind a reduced amount of solid residue after evaporation is disclosed. The acidic electrolyzed water can have an effective chlorine concentration of 10 ppm or more, and contain metal ions at a concentration (molar equivalent ratio) of from 0.46 to 1.95 relative to the effective chlorine concentration, the metal ions being cations of an alkali metal or alkaline-earth metal.

### RELATED APPLICATIONS

This application claims priority to Japanese Application No. 2014-094442, filed May 1, 2014, which is incorporated herein by reference in its entirety.

### TECHNICAL FIELD

The present application relates to acidic electrolyzed water and a manufacturing method therefor, a disinfectant and a cleanser containing acidic electrolyzed water, a disinfecting method using acidic electrolyzed water, and a manufacturing device for acidic electrolyzed water.

### BACKGROUND ART

Acidic electrolyzed water is obtained by electrolyzing a solution of water and electrolytes such as sodium chloride and hydrochloric acid. Acidic electrolyzed water having a pH value of 2.7 or less is generally referred to as “strongly acidic water” and is known to have a strong disinfecting effect (see PCT Application No. PCT/JP1995/001503). However, strongly acidic water maintains its disinfecting power for only a short period of time and cannot be stored for a very long period of time.

Because acidic electrolyzed water contains electrolytes, these electrolytes are left behind as a solid residue when acidic electrolyzed water evaporates. For example, when acidic electrolyzed water is used to clean components, a solid residue sometimes remains on the clean component after it has dried.

Humidifiers are commonly used to suppress the functions of viruses (such as influenza viruses). Thus, a method is desired which is able to more effectively suppress the functions of viruses when a humidifier is used.

Tap water is commonly used in humidifiers. However, (chalky) solids gradually build up on components inside the humidifier such as the filter when tap water is used. In order to maintain humidifier performance, the removal of these solids is desired. Unfortunately, these solids can be very difficult to remove.

### SUMMARY OF THE INVENTION

In one embodiment an acidic electrolyzed water having an effective chlorine concentration of 10 ppm or more, and containing metal ions at a concentration (molar equivalent ratio) of from 0.46 to 1.95 relative to the effective chlorine concentration, the metal ions being cations of an alkali metal or alkaline-earth metal. In the acidic electrolyzed water according to 1, the pH value can be from 3.0 to 7.0. In the acidic electrolyzed water according to 1 or 2, the solid content can be 300 ppm or less.

In the acidic electrolyzed water the alkali metal can be potassium or sodium and the alkaline-earth metal can be calcium or magnesium. A cleanser or a disinfectant containing acidic electrolyzed water as discussed above can be provided. A method for disinfecting microbes contained in air can include a step of evaporating in the air acidic electrolyzed water as discussed above.

Another aspect of the disclosure is a method for manufacturing acidic electrolyzed water, the method comprising a step of electrolyzing raw acidic electrolyzed water having an effective chlorine concentration of 10 ppm or more, and containing metal ions at a concentration (molar equivalent ratio) of from 1.23 to 2.54 relative to the effective chlorine concentration (where the metal ions are cations of an alkali metal or alkaline-earth metal).

In a method for manufacturing acidic electrolyzed water the acidic electrolyzed water can be obtained from the step of electrolyzing raw acidic electrolyzed water.

The method for manufacturing acidic electrolyzed water can also include, prior to the step of electrolyzing raw acidic electrolyzed water, a step of preparing raw acidic electrolyzed water by electrolyzing raw water containing a predetermined concentration of the metal ions and a chlorine-based electrolyte aqueous solution via an anion-exchange membrane.

The method for manufacturing acidic electrolyzed water can also include, prior to the step of electrolyzing raw acidic electrolyzed water, the steps of preparing primary electrolyzed water by electrolyzing raw water and chlorine-based electrolyte aqueous solution via an anion-exchange membrane, and preparing raw acidic electrolyzed water by adding the metal ions to the primary electrolyzed water.

Another embodiment of the application is a device for manufacturing acidic electrolyzed water comprising: a primary electrolysis bath for obtaining raw acidic electrolyzed water by electrolyzing raw water containing a predetermined concentration of metal ions (where the metal ions are cations of an alkali metal or alkaline-earth metal), and a secondary electrolysis bath for obtaining secondary electrolyzed water by electrolyzing the raw acidic electrolyzed water; the primary electrolysis bath comprising: an anode chamber containing an anode, a cathode chamber containing a cathode, and a middle chamber provided between the anode chamber and the cathode chamber, an anion-exchange membrane being provided between the anode chamber and the middle chamber, a cation-exchange membrane being provided between the cathode chamber and the middle chamber, the raw water containing metal ions being introduced to the anode chamber, raw water being introduced to the cathode chamber, and the chlorine-based electrolyte aqueous solution being introduced to the middle chamber, the raw acidic electrolyzed water being generated in the anode chamber.

In a device for manufacturing acidic electrolyzed water, alkaline water containing the metal ions can be generated in the cathode chamber, and a means can be provided for adding the alkaline water generated in the cathode chamber to raw water prior to the introduction of raw water containing the metal ions to the anode chamber.

A device for manufacturing acidic electrolyzed water can also include a means for adding the metal ions to raw water provided prior to the introduction of the raw water containing metal ions to the anode chamber.

Another embodiment of the application is a device for manufacturing acidic electrolyzed water comprising: a primary electrolysis bath for obtaining primary electrolyzed water by electrolyzing raw water and a chlorine-based electrolyte aqueous solution, and a secondary electrolysis bath for obtaining secondary electrolyzed water by electrolyzing raw acidic electrolyzed water prepared by adding a predetermined concentration of metal ions (where the metal ions are cations of an alkali metal or alkaline-earth metal) to the primary electrolyzed water; the primary electrolysis bath comprising: an anode chamber containing an anode, a cathode chamber containing a cathode, and a middle chamber provided between the anode chamber and the cathode chamber, a cation-exchange membrane being provided between the cathode chamber and the middle chamber, an anion-exchange membrane being provided between the middle chamber and the anode chamber, raw water being introduced to the anode chamber and the cathode chamber, and the chlorine-based electrolyte aqueous solution being introduced to the middle chamber, the primary electrolyzed water being generated in the anode chamber. The device for manufacturing acidic electrolyzed water can also include a means for adding the metal ions to the primary electrolyzed water, raw acidic electrolyzed water being obtained by the means for adding the metal ions. The device for manufacturing acidic electrolyzed water can be configured so that alkaline water containing metal ions can be generated in the cathode chamber, a means can be provided for adding the alkaline water to the primary electrolyzed water prior to the introduction of the raw acidic electrolyzed water to the secondary electrolysis bath, and the raw acidic electrolyzed water can be obtained by the means for adding the alkaline water.

The acidic electrolyzed water in any of 1 through 5 has an effective chlorine concentration of 10 ppm or more, and contains metal ions at a concentration (molar equivalent ratio) of from 0.46 to 1.95 relative to the effective chlorine concentration, the metal ions being cations of an alkali metal or alkaline-earth metal. The presence of cations of these metals can render the pH value of the acidic electrolyzed water in the present embodiment acidic (for example, a pH value from 3 to 7). At the same time, the presence of cations of these metals can suppress side reactions at the cathode during electrolysis. Because this can suppress consumption of HClO, the disinfecting effect of the acidic electrolyzed water can be increased. Also, because of the acidity (for example, a pH from 3 to 7), it has disinfecting power over a long period of time and, thus, can be stored for a long period of time. The amount of solids left over after evaporation is also reduced. As a result, the burden on living tissue is reduced, safety is improved, and the impact on the environment is reduced.

Because the acidic electrolyzed water can maintain its disinfecting power even when not stored in a dark place to avoid exposure to direct sunlight, it is easy to store. As a result, the acidic electrolyzed water makes for a particularly good disinfectant or cleaner.

Because the method for disinfecting microbes contained in air described above includes a step of evaporating the acidic electrolyzed water in the air, microbes contained in air can be effectively eliminated.

The method for manufacturing acidic electrolyzed water includes a step of electrolyzing raw acidic electrolyzed water having an effective chlorine concentration of 10 ppm or more, and containing metal ions at a concentration (molar equivalent ratio) of from 1.23 to 2.54 relative to the effective chlorine concentration. The result is efficient electrolysis and disinfecting power that lasts for a long time. The resulting acidic electrolyzed water can be stored over a long period of time, and leaves behind less solid residue after evaporation.

The device for manufacturing acidic electrolyzed water provides efficient electrolysis and disinfecting power that lasts for a long time. The resulting acidic electrolyzed water can be stored over a long period of time, and leaves behind less solid residue after evaporation.

### BRIEF DESCRIPTION OF THE DRAWINGS

FIG. 1 is the chemical equilibrium equation for the acidic electrolyzed water in an embodiment of the present invention.

FIG. 2(A) is a diagram used to schematically illustrate an embodiment of a manufacturing device for acidic electrolyzed water.

FIG. 2(B) is a diagram used to schematically illustrate another embodiment of a manufacturing device for acidic electrolyzed water.

FIG. 2(C) is a diagram used to schematically illustrate another embodiment of a manufacturing device for acidic electrolyzed water.

FIG. 2(D) is a diagram used to schematically illustrate another embodiment of a manufacturing device for acidic electrolyzed water.

FIG. 3 is a graph showing the relationship between the effective chlorine concentration of the primary electrolyzed water and the applied current value in the secondary electrolytic step in an embodiment of the present invention.

FIG. 4 is a graph showing the relationship between the sodium ion concentration and the change in the effective chlorine concentration over time in acidic electrolyzed water in an embodiment of the present invention.

FIG. 5 is a graph showing the relationship between the sodium ion concentration in and the pH of acidic electrolyzed water in an embodiment of the present invention.

FIG. 6 is a graph showing the relationship between the initial effective chlorine concentration and the pH in the secondary electrolytic step in an embodiment of the present invention.

FIG. 7 is a graph showing the relationship between the initial effective chlorine concentration and the effective chlorine concentration in the secondary electrolytic step in an embodiment of the present invention.

FIG. 8 is a graph showing the change over time in the effective chlorine concentration when acidic electrolyzed water in an embodiment of the present invention is stored openly.

FIG. 9 is a graph showing the relationship between each type of electrolyte included in acidic electrolyzed water in equivalent amounts and the pH of the acid electrolyzed water in an example of the present invention.

FIG. 10 is a graph showing the relationship between each type of electrolyte included in acidic electrolyzed water in equivalent amounts and the effective chlorine concentration of the acid electrolyzed water in an example of the present invention.

FIG. 11 is a diagram used to schematically illustrate the method in a disinfecting test conducted on airborne microbes using acidic electrolyzed water in an example of the present invention.

FIG. 12 is a series of photographs of Petri dishes showing the results of the disinfecting test conducted on airborne microbes using acidic electrolyzed water in an example of the present invention.

FIG. 13 is a graph showing the relationship between electrolysis time, pH and effective chlorine composition when electrolysis is performed with 3 mass % hydrochloric acid in a comparative example of the present invention.

FIG. 14 is a graph showing the relationship between electrolysis time, pH and effective chlorine composition when electrolysis is performed with dilute hydrochloric acid (0.008 mass % hydrochloric acid) in a comparative example of the present invention.

### DETAILED DESCRIPTION OF THE PREFERRED EMBODIMENTS

The following is a more detailed explanation of the present invention with reference to the drawings. In the present invention, “parts” refers to “parts by mass” unless otherwise indicated. One aspect of the disclosure is to provide acidic electrolyzed water and a manufacturing method therefor, a disinfectant and a cleanser containing acidic electrolyzed water, and a disinfecting method using acidic electrolyzed water which has disinfecting power for a long period of time, and which leaves behind a reduced amount of solid residue after evaporation. Applicants have discovered that disinfecting power could be maintained over a long period of time, and the amount of solid residue left behind after evaporation could be reduced by using certain electrolytes.

The present embodiment is acidic electrolyzed water having an effective chlorine concentration of 10 ppm or more, and containing metal ions at a concentration (molar equivalent ratio) of from 0.46 to 1.95 relative to the effective chlorine concentration, the metal ions being cations of an alkali metal or alkaline-earth metal.

The acidic electrolyzed water in the present embodiment has an effective chlorine concentration of 10 ppm or more, preferably of 20 ppm or more, and usually 1,000 ppm or less in order to exhibit sufficient disinfecting power. In the present invention, the effective chlorine concentration of the acidic electrolyzed water can be measured using a commercially available chlorine concentration measuring device.

The metal ions included in the acidic electrolyzed water of the present embodiment are cations of an alkali metal or alkaline-earth metal. Examples of alkali metals include lithium, sodium, and potassium. Sodium or potassium is preferred. Examples of alkaline-earth metals include magnesium and calcium. Calcium is preferred.

In the present invention, the molar equivalent ratio concentration of metal ions relative to the effective chlorine concentration, on condition that the effective chlorine concentration is 1 mol/L, is 1 when (1) the metal is monovalent (for example, an alkali metal) and the molar concentration of metal ions is 1 mol/L, and 1 when (2) the metal is divalent (for example, an alkaline-earth metal) and the molar concentration of metal ions is 0.5 mol/L.

In the acidic electrolyzed water of the present embodiment, the pH of the acidic electrolyzed water is too low when the molar equivalent ratio concentration of metal relative to the effective chlorine concentration is less than 0.46, and the acidic electrolyzed water becomes basic when the molar equivalent ratio concentration of metal relative to the effective chlorine concentration is greater than 1.95. This also causes instability and increases the solid content of the acidic electrolyzed water. The pH value of the acidic electrolyzed water of the present embodiment can be from 3.0 to 7.0. From the standpoint of less solid content in the acidic electrolyzed water, a metal ion concentration (molar equivalent ratio) relative to the effective chlorine concentration from 0.46 to 1.95 is preferred.

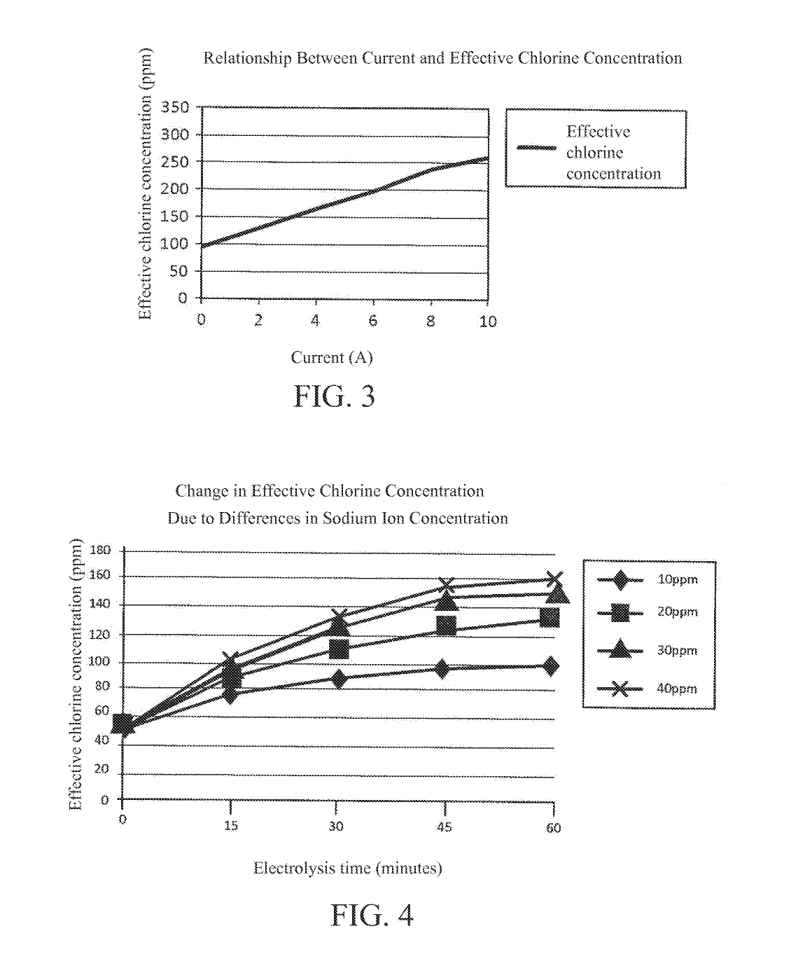

FIG. 3 is a graph showing the relationship between the effective chlorine concentration of the primary electrolyzed water and the applied current value in the present embodiment. As shown in FIG. 3, the effective chlorine concentration of the acidic electrolyzed water in the present embodiment depends on the value of the current applied during electrolysis. The effective chlorine composition of the acidic electrolyzed water generally rises when the current value is increased.

Because the concentration of metal ions in acidic electrolyzed water of the present embodiment is not changed much by electrolysis, the acidic electrolyzed water of the present embodiment can be kept acidic only if the effective chlorine concentration of the acidic electrolyzed water ranges from 0.46 to 1.95 (molar equivalent ratio).

In the acidic electrolyzed water of the present embodiment, the metal ion content is usually from 0.0001 ppm to 1,000 ppm (preferably from 0.001 ppm to 500 ppm). From the standpoint of less solid content, it is more preferably 300 ppm or less.

The metal ions may be added to the raw acidic electrolyzed water in the form of a hydroxide, carbonate salt, or bicarbonate salt of an alkali metal or alkaline-earth metal.

In the present invention, hydroxides are compounds containing hydroxide ions (OH⁻), carbonate salts are compounds containing carbonate ions (CO₃²⁻), and bicarbonate salts are compounds containing bicarbonate ions (HCO₃⁻).

In other words, hydroxides, carbonate salts, and bicarbonate salts of alkali metals and alkaline-earth metals are electrolytes composed of anions produced by water and/or carbon dioxide, and metal ions (cations) of alkali metals or alkaline-earth metals. Acidic electrolyzed water of the present embodiment can be obtained by electrolyzing an aqueous solution containing chlorine ions, these anions, and these cations.

Here, hydroxides of alkali metals include sodium hydroxide and potassium hydroxide, carbonate salts of alkali metals include sodium carbonate and potassium carbonate, and bicarbonate salts of alkali metals include sodium bicarbonate and potassium bicarbonate. These can be used alone or in combinations of two or more. These hydroxides, carbonate salts, and bicarbonate salts of alkali metals, when used in applications such as medicines, food products, and cosmetics, are safe and do not harm the environment.

Here, hydroxides of alkaline-earth metals include calcium hydroxide and magnesium hydroxide, carbonate salts of alkaline-earth metals include calcium carbonate and magnesium carbonate, and bicarbonate salts of alkaline-earth metals include calcium bicarbonate and magnesium bicarbonate. These hydroxides, carbonate salts, and bicarbonate salts of alkali metals, when used in applications such as medicines, food products, and cosmetics, are safe and do not harm the environment.

The pH value of the acidic electrolyzed water in the present embodiment is preferably 7.0 or less, and more preferably from 3.0 to 7.0, in order to stabilize the acidic electrolyzed water and inhibit the production of trihalomethanes. In the present invention, the pH value of the acidic electrolyzed water can be measured using a commercially available pH measuring device.

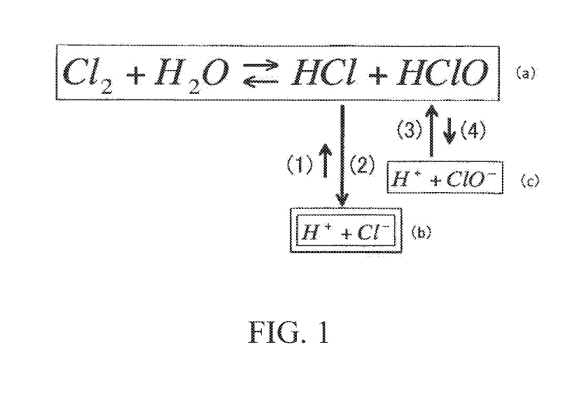

FIG. 1 is the chemical equilibrium equation in the acidic electrolyzed water of the present invention. Equation (a) in FIG. 1 maintains the equilibrium in the acidic electrolyzed water of the present invention. Hydrochloric acid (HCl) maintains the equilibrium in the directions of arrow (1) and arrow (2) between Equation (a) in FIG. 1 and Equation (b) in FIG. 1, and hypochlorous acid (HClO) maintains the equilibrium in the directions of arrow (3) and arrow (4) between Equation (a) in FIG. 1 and Equation (c) in FIG. 1. Because hydrochloric acid (HCl) is a very strong acid, it is easy to ionize and arrow (2) predominates. Because hypochlorous acid (HClO) is affected by hydrogen chloride, it is hardly ionized at all and arrow (3) predominates.

Because the acidic electrolyzed water in the present embodiment has an effective chlorine concentration of 10 ppm or more, and contains metal ions at a concentration (molar equivalent ratio) of from 0.46 to 1.95 relative to the effective chlorine concentration, side reactions can be suppressed at the cathode during electrolysis. Because this can suppress consumption of HClO, the disinfecting effect of the acidic electrolyzed water can be maintained.

Because the concentration of HClO is maintained in the acidic electrolyzed water of the present embodiment, superior disinfecting power can be expected.

The chlorine-based electrolyte content of the acidic electrolyzed water in the present embodiment is preferably 0.1 mass % or less, more preferably 0.05 mass % or less, and even more preferably 0.025 mass % or less, in terms of sodium chloride in order to prevent corrosion of metal and the escape of chlorine gas from the acidic electrolyzed water in the present embodiment.

When the (added) chlorine-based electrolyte content of the acidic electrolyzed water in the present embodiment exceeds 0.1 mass % in terms of sodium chloride, the chloride ions bond with the hydrogen ions in the acidic electrolyzed water. As a result, the equilibrium between Equation (a) and Equation (b) in FIG. 1 is biased in the direction of arrow (1), and the equilibrium of Equation (a) in FIG. 1 is biased to the left. Consequently, the chloride ions are released as chlorine, the effective chlorine concentration of the acidic electrolyzed water is lowered, and the disinfecting effect is reduced.

In the present invention, “chlorine-based electrolyte” refers to an electrolyte that produces chloride ions when dissolved in water. Chlorine-based electrolytes include chlorides of alkali metals (such as sodium chloride and potassium chloride), and chlorides of alkaline rare earth metals (such as calcium chloride and magnesium chloride).

The acidic electrolyzed water in the present embodiment can be used as a disinfectant and/or cleanser in various fields such as medicine, veterinary medicine, food processing, and manufacturing. It can be used to clean and disinfect tools and affected areas in medicine and veterinary medicine. The acidic electrolyzed water in the present embodiment is not unpleasant to use because it lacks a pungent odor such as the odor of halogens.

Because the acidic electrolyzed water in the present embodiment is very stable, it can be placed in a container and used as acidic electrolyzed water inside the container.

Also, by evaporating acidic electrolyzed water of the present embodiment in air, airborne microbes can be killed. More specifically, by using acidic electrolyzed water of the present invention as the water in a humidifier, airborne microbes can be effectively killed.

Because the acidic electrolyzed water of the present embodiment has an effective chlorine concentration of 10 ppm or more, and contains metal ions at a concentration (molar equivalent ratio) of from 0.46 to 1.95 relative to the effective chlorine concentration (where the metal ions are cations of an alkali metal or alkaline-earth metal), the metal ions being cations of an alkali metal or alkaline-earth metal, electrolysis renders the electrolyzed water acidic (for example, a pH value from 3 to 7) and side reactions at the cathode are suppressed, thereby suppressing consumption of HClO. Also, because of the acidity (for example, a pH from 3 to 7), the acidic electrolyzed water of the present embodiment has disinfecting power over a long period of time and, thus, can be stored for a long period of time. The amount of solids left over after evaporation is also reduced.

In other words, the acidic electrolyzed water of the present embodiment has a metal ion concentration in a range corresponding to the effective chlorine concentration. When the effective chlorine concentration in the acidic electrolyzed water of the present embodiment is low (for example, from 10 ppm to 80 ppm), the metal ion concentration is as low as the effective chlorine concentration in a relative sense. When the effective chlorine concentration in the acidic electrolyzed water of the present embodiment is high (for example, from 100 ppm), the metal ion concentration is also higher. However, this can be diluted with water before use.

In particular, when the metal ions are derived from cations (metal ions) of a hydroxide, carbonate salt, or bicarbonate salt of an alkali metal or alkaline-earth metal, hydroxide ions (OH⁻) constituting the hydroxide, carbonate ions (CO₃²⁻) constituting the carbonate salt, and bicarbonate ions (HCO₃⁻) constituting the bicarbonate salt are derived. When the water content of the acidic electrolyzed water of the present embodiment is evaporated, water and/or gas (for example, carbon dioxide) is produced, and the solid residue left behind after the water content has evaporated is reduced.

As a result, the burden on living tissue is reduced, safety is improved, and the impact on the environment is reduced. Because the acidic electrolyzed water maintains its disinfecting power even when not stored in a dark place to avoid exposure to direct sunlight, it is easy to store.

An indicator of the long-term disinfecting power of the acidic electrolyzed water of the present invention is a residual chlorine concentration of 10 ppm or more, and preferably 20 ppm or more after the acidic electrolyzed water has been allowed to stand for 14 days in open air at a temperature of 22° C. and a humidity of 40%.

As an indicator of how little solid content is included in the acidic electrolyzed water of the present embodiment, the acidic electrolyzed water of the present embodiment can have a solid content of 300 ppm or less. Here, the solid content of the acidic electrolyzed water of the present embodiment is the mass of residue after 20 ml of the acidic electrolyzed water has been exposed to air for 48 hours at a temperature of 60° C. and a humidity of 30%.

(2) When inorganic substances such as organic acids and salts of organic acids are present in acidic electrolyzed water, the organic substances are usually oxidized by chlorine and the chlorine is consumed. This reduces the disinfecting power of the acidic electrolyzed water. Because the metal ions in the acidic electrolyzed water of the present embodiment are not organic substances, they are not oxidized by chlorine. As a result, the disinfecting power of the acidic electrolyzed water is maintained over a long period of time.

The method for manufacturing acidic electrolyzed water in one embodiment of the present invention includes a step of electrolyzing raw acidic electrolyzed water having an effective chlorine concentration of 10 ppm or more, and containing metal ions at a concentration (molar equivalent ratio) of from 1.23 to 2.54 relative to the effective chlorine concentration (where the metal ions are cations of an alkali metal or alkaline-earth metal).

Here, the step of electrolyzing the raw acidic electrolyzed water corresponds to the electrolyzing of the raw acidic electrolyzed water in the second electrolysis bath (second electrolyzing step) in the manufacturing device for acidic electrolyzed water in the embodiment described below. The step of electrolyzing the raw acidic electrolyzed water produces the acidic electrolyzed water (secondary electrolyzed water) in the embodiment described above.

In the manufacturing method for acidic electrolyzed water of the present embodiment, the target of electrolysis in the first electrolyzing step is raw water and chlorine-based electrolyte aqueous solution.

In the present invention, “raw water” is water having a total electrolyte concentration of 15 ppm or less. For example, the metal ion concentration (sodium ion concentration) in raw water can be 2 ppm or less, and preferably 1 ppm or less. The raw water can be ion-exchanged water, distilled water, or RO water.

In the primary electrolyzing step, raw acidic electrolyzed water can be prepared using either one of the two methods in (1) and (2) below. The step of electrolyzing the primary electrolyzed water (the primary electrolyzing step) corresponds to the electrolyzing of the primary electrolyzed water in the first electrolysis bath in the manufacturing device for acidic electrolyzed water in the embodiment described below.

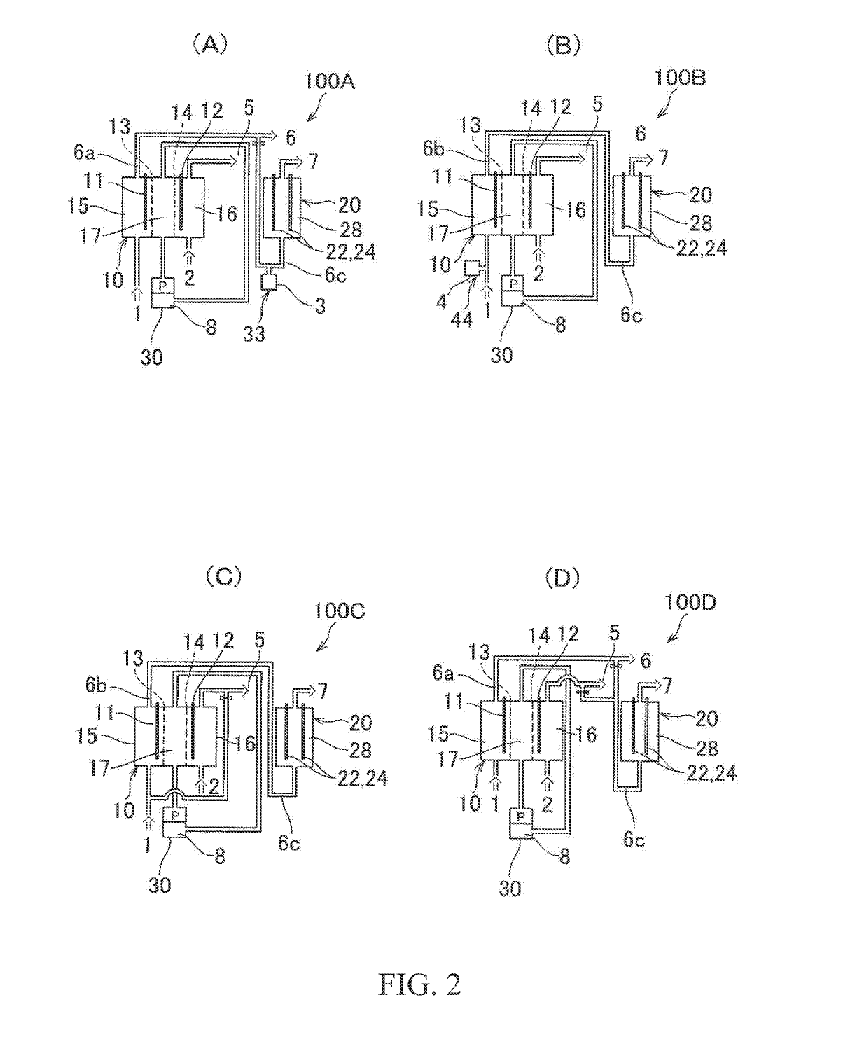

For example, prior to the secondary electrolyzing step (the step in which the raw acidic electrolyzed water is electrolyzed), the raw acidic electrolyzed water can be prepared by electrolyzing raw water containing metal ions at a predetermined concentration (metal ions at a concentration (molar equivalent ratio) of from 1.23 to 2.54 relative to the effective chlorine concentration) and a chlorine-based electrolyte aqueous solution (for example, in FIG. 2 (B) and FIG. 2 (C) described below). Here, the raw water and chlorine-based electrolyte aqueous solution are electrolyzed via an anion-exchange membrane to produce the raw acidic electrolyzed water in the chamber receiving the raw water (the cathode chamber 15 in FIG. 2 (B) and FIG. 2 (C)).

For example, prior to the step in which the raw acidic electrolyzed water is electrolyzed (the secondary electrolyzing step), the raw acidic electrolyzed water can be prepared by electrolyzing raw water and a chlorine-based electrolyte solution to obtain primary electrolyzed water, and then adding metal ions to the primary electrolyzed water to obtain a concentration (molar equivalent ratio) of from 1.23 to 2.54 relative to the effective chlorine concentration (for example, in FIG. 2 (A) and FIG. 2 (D) described below). Here, the raw water and chlorine-based electrolyte aqueous solution are electrolyzed via an anion-exchange membrane to produce primary electrolyzed water in the chamber receiving the raw water (the cathode chamber 15 in FIG. 2 (A) and FIG. 2 (D)).

The primary electrolyzed water can be prepared by performing electrolysis while housing chlorine-based electrolyte aqueous solution in the anode chamber and cathode chamber using a water electrolyzing device having a structure in which the anode chamber and the cathode chamber are partitioned by a partitioning membrane (a two-bath water electrolyzing device), or by performing electrolysis while housing a high-concentration of chlorine-based electrolyte aqueous solution in a middle chamber using a water electrolyzing device having a structure in which the anode chamber and the middle chamber and the middle chamber and the cathode chamber are partitioned by two partitioning membranes (a three-bath water electrolyzing device such as the acidic electrolyzed water manufacturing device described below in FIG. 2 (A), FIG. 2 (B), FIG. 2 (C), and FIG. 2 (D)).

When a two-bath water electrolyzing device is used to perform the electrolysis, the concentration of chlorine-based electrolyte aqueous solution is preferably from 0.1 mass % to 0.2 mass %. When a three-bath water electrolyzing device is used to perform the electrolysis, the concentration of the high-concentration chlorine-based electrolyte aqueous solution should be as high as possible while also not adversely affecting the properties of the primary electrolyzed water.

From the standpoint of having a low concentration of electrolytes in the primary electrolyzed water, the primary electrolyzed water is preferably prepared using a three-bath water electrolyzing device. When a two-bath water electrolyzing device is used to prepare the primary electrolyzed water, the concentration of electrolytes in the primary electrolyzed water produced by the two-bath water electrolyzing device can be lowered by adding pure water (for example, distilled water or ion-exchanged water) to the produced electrolyzed water.

The primary electrolyzed water may be prepared using the water electrolyzing device described above. Because such water electrolyzing devices are commercially available as electrolyzed water manufacturing devices, a commercially available electrolyzed water manufacturing device can also be used to prepare the primary electrolyzed water.

Examples of commercially available water electrolyzing devices include the Excel-FX (MX-99) from Nambu Co., Ltd., the ROX-10WB3 from Hoshizaki Denki Co., Ltd., the α-Light from Amano Co., Ltd., and the ESS-Zero from Shinsei Co., Ltd. The primary electrolyzed water can be manufactured using any commercially available electrolyzed water manufacturing device. The primary acidic electrolyzed water can also be manufactured using the electrolyzed water manufacturing method described in JP2001-286868A.

In the acidic electrolyzed water of the present embodiment, in order to obtain a molar equivalent ratio concentration of metal ions relative to the effective chlorine concentration of from 0.46 to 1.95, the amount of metal ions added to primary electrolyzed water having an effective chlorine concentration of 50 ppm is from 20 ppm to 41 ppm. For example, when the metal ions added to primary electrolyzed water having an effective chlorine concentration of 50 ppm are sodium ions, the amount of sodium is preferably from 20 ppm to 41 ppm, and more preferably from 21 ppm to 40 ppm. When the metal ions are potassium ions, the amount of potassium is preferably from 20 ppm to 41 ppm, and more preferably from 21 ppm to 40 ppm. When the metal ions are calcium ions, the amount of calcium is preferably from 10 ppm to 20.5 ppm, and more preferably from 11 ppm to 19.5 ppm. When the metal ions are magnesium ions, the amount of magnesium is preferably from 10 ppm to 20.5 ppm, and more preferably from 11 ppm to 19.5 ppm.

The manufacturing method for acidic electrolyzed water of the present embodiment includes a step in which raw acidic electrolyzed water having an effective chlorine concentration of 10 ppm or less and metal ions at a predetermined concentration (metal ions at a concentration (molar equivalent ratio) of from 1.23 to 2.54 relative to the effective chlorine concentration) is electrolyzed.

The secondary electrolysis step can be performed in the secondary electrolysis bath 20 of the manufacturing device for acidic electrolyzed water depicted in FIG. 2 (A), FIG. 2 (B), FIG. 2 (C), and FIG. 2 (D) and explained below.

The method for manufacturing acidic electrolyzed water of the present embodiment has both a primary electrolysis step and a secondary electrolysis step. For example, it is difficult to obtain secondary electrolyzed water (acidic electrolyzed water) having an effective chlorine concentration of 10 ppm or more, metal ions at a concentration (molar equivalent ratio) of from 0.46 to 1.95 relative to the effective chlorine concentration, and acidity (a pH from 3 to 7) when the primary electrolysis step is performed for a long period of time. This is because the chlorine ions in the electrolyzed water are consumed as chlorine and the effective chlorine concentration decreases when the primary electrolysis step is takes a long time.

In the method for manufacturing acidic electrolyzed water of the present embodiment, when electrolysis is performed on the raw acidic electrolyzed water in the secondary electrolysis step, the raw acidic electrolyzed water undergoing electrolysis include electrolytes in order to obtain the secondary electrolyzed water. In other words, the chlorine ions in the raw acidic electrolyzed water are consumed in the secondary electrolysis step. As a result, the concentration of chlorine ions in the secondary electrolyzed water is lower than the concentration of chlorine ions in the raw acidic electrolyzed water. Because the metal ions are susceptible to ionization, metal ions continue to be present in the electrolysis bath. As a result, the concentration of metal ions in the secondary electrolyzed water is substantially unchanged relative to the concentration of metal ions in the raw acidic electrolyzed water. As a result, the chlorine ion concentration is reduced while the metal ion concentration remains substantially unchanged, resulting in secondary electrolyzed water having very little solid content.

FIG. 2 (A), FIG. 2 (B), FIG. 2 (C), and FIG. 2 (D) are diagrams used to schematically illustrate the manufacturing device for acidic electrolyzed water in an embodiment of the present invention. Acidic electrolyzed water manufacturing devices 100A, 100B, 100C, and 100D each include a primary electrolysis bath 10 in which electrolysis is performed on the primary electrolyzed water (primary electrolysis step) and a secondary electrolysis bath 20 in which electrolysis is performed on the raw acidic electrolyzed water (secondary electrolysis step) to obtain secondary electrolyzed water (the acidic electrolyzed water of the present embodiment).

The primary electrolysis bath 10 includes an anode chamber 15 containing an anode 11, a cathode chamber 16 containing a cathode 12, and a middle chamber 17 provided between the anode chamber 15 and the cathode chamber 16. An anion-exchange membrane 13 is provided between the anode chamber 15 and the middle chamber 17, and a cation-exchange membrane 14 is provided between the cathode chamber 16 and the middle chamber 17. The primary electrolysis bath 10 can be one of the commercially available electrolyzed water manufacturing devices mentioned in Section 2.1. The secondary electrolysis bath 20 includes electrodes 22, 24, and a reaction chamber 28.

In the acidic electrolyzed water manufacturing devices 100A, 100B, 100C, 100D, raw water 1, 2 is introduced to the anode chamber 15 and the cathode chamber 16, and a chlorine-based electrolyte aqueous solution is introduced to the middle chamber 17. The primary electrolyzed water 6 is generated in the anode chamber 15 of the primary electrolyte bath 10.

In the acidic electrolyzed water manufacturing devices 100A, 100B, 100C, and 100D shown in FIG. 2 (A), FIG. 2 (B), FIG. 2 (C), and FIG. 2 (D), the following reactions occur at the anode 11 and the cathode 12 of the primary electrolysis bath 10, and the electrodes 22, 24 of the secondary electrolysis bath 20.

[Reactions at the Anode]

2Cl⁻→Cl₂+2e⁻ (i) (main reaction)

4OH⁻→O₂+2H₂O+4e⁻ (ii) (side reaction)

[Reactions at the Cathode]

2H⁺+2e⁻→H₂ (iii) (main reaction)

H⁺+2e⁻+HClO→2H₂O+Cl⁻ (iv) (side reaction)

The disinfecting power of acidic electrolyzed water is derived from hypochlorous acid (HClO) (Equation (a) in FIG. 1). The chlorine in the hypochlorous acid readily evaporates as it is a gas at normal temperatures. As a result, the disinfecting power of acidic electrolyzed water gradually diminishes as chlorine is lost.

In the method for manufacturing acidic electrolyzed water of the present embodiment, a creative idea is used to suppress the loss of chlorine. The equilibrium in Equation (a) of FIG. 1 is biased to the right by reducing the amount of HCl, and the concentration of hypochlorous acid (HClO) is increased.

The reduction in HCl is one factor in the rise of the pH of the acidic electrolyzed water. In order to counter this, the method for manufacturing acidic electrolyzed water of the present embodiment suppresses the rise in pH while increasing the concentration of hypochlorous acid (HClO).

In the method for manufacturing acidic electrolyzed water of the present embodiment, raw electrolyzed water having an effective chlorine concentration of 10 ppm or more and metal ions (cations) at a predetermined concentration (a metal ion concentration (molar equivalent ratio) of from 1.23 to 2.54 relative to the effective chlorine concentration) is electrolyzed in the secondary electrolysis bath 20 of the acidic electrolyzed water manufacturing devices 100A, 100B, 100C, and 100D shown in FIG. 2 (A), FIG. 2 (B), FIG. 2 (C), and FIG. 2 (D). The presence of cations easily converts hydrogen atoms (H⁺), which are less susceptible to ionization than cations, into hydrogen (H₂) (Equation (iii) progresses to the right). This can improve electrolysis efficiency.

Because the Cl⁻ generated in Equation (iv) is also converted to Cl₂, the equilibrium moves from Equation (a) in FIG. 1 towards Equation (b) in FIG. 1 as the amount of Cl⁻ is reduced, and H⁺ and Cl⁻ is produced from HCl. In this way, the equilibrium in Equation (a) of FIG. 1 becomes biased to the right. As a result, the amount of hypochlorous acid (HClO) in the final acidic electrolyzed water of the present embodiment can be increased.

In the second electrolytic step, when acidic electrolyzed water having an effective chlorine concentration of 10 ppm or more and a metal ion concentration (molar equivalent ratio) of less than 1.23 relative to the effective chlorine concentration is electrolyzed, the concentration of metal ions is low and the electrolysis does not progress adequately.

The acidic electrolyzed water manufacturing device 100A in FIG. 2 (A) includes a primary electrolysis bath 10 and a secondary electrolysis bath 20. The primary electrolysis bath 10 includes an anode chamber 15, a cathode chamber 16, and a middle chamber 17. The anode chamber 15 includes an anode 11, and the cathode chamber 16 includes a cathode 12. An anion-exchange membrane 13 is provided between the anode chamber 15 and the middle chamber 17 to allow anions to pass between the anode chamber 15 and the middle chamber 17. A cation-exchange membrane 13 is provided between the middle chamber 17 and the cathode chamber 16 to allow cations to pass between the middle chamber 17 and the cathode chamber 16.

Raw water 1 is introduced to the anode chamber 15, and raw water 2 is also introduced to the cathode chamber 16. A chlorine-based electrolyte (for example, sodium chloride) aqueous solution 8 is introduced to the middle chamber 17, and the chlorine-based electrolyte aqueous solution 8 is circulated inside the middle chamber 17 using a pump 30. When the chlorine-based electrolyte in the chlorine-based electrolyte aqueous solution 8 is sodium chloride, the concentration of sodium chloride in the chlorine-based electrolyte aqueous solution 8 is preferably 26 mass % or less.

In the acidic electrolyzed water manufacturing device 100A, as shown in FIG. 2 (A), electrolysis is performed in the primary electrolysis bath 10 (primary electrolysis step), and the primary electrolyzed water 6a is produced in the anode chamber 15.

Also, the acidic electrolyzed water manufacturing device 100A includes, as shown in FIG. 2 (A), a means for adding cations (metal ions) of an alkali metal or alkaline-earth metal to the primary electrolyzed water 6a (adding device 33).

More specifically, the adding device 33 adds alkaline water 3 containing metal ions to the primary electrolyzed water 6a. Using this adding device 33, raw acidic electrolyzed water 6c is obtained which has an effective chlorine concentration of 10 ppm or more, and containing metal ions at a predetermined concentration (a metal ion concentration (molar equivalent ratio) of from 1.23 to 2.54 relative to the effective chlorine concentration).

Next, the raw acidic electrolyzed water 6c is introduced to the secondary electrolysis bath 20 and electrolysis is performed on the raw acidic electrolyzed water 6c in the secondary electrolysis bath 20 (secondary electrolysis step) to obtain secondary electrolyzed water 7.

Here, in order to reduce the solid content of the secondary electrolyzed water 7, the metal ions of an alkali metal or alkaline-earth metal included in alkaline water 3 (or alkaline water 4 and 5 described below) are preferably metal ions (cations) derived from a hydroxide, carbonate salt, or bicarbonate salt of an alkali metal or alkaline-earth metal. The hydroxide, carbonate salt, or bicarbonate salt of an alkali metal or alkaline-earth metal can be any one of the examples mentioned in Section 1.2.

The anode 11 can be made, for example, of indium oxide or platinum. The cathode 12 is preferably made of a metal that is not susceptible to ionization by hydrogen atoms. Examples include platinum electrodes and diamond electrodes.

In order to obtain the acidic electrolyzed water of the present embodiment, the current supplied to the electrodes (anode 11 and cathode 12) of the primary electrolysis bath 10 and the electrodes 22, 24 of the secondary electrolysis bath 20 is preferably 1 A or more.

Electrolysis is performed in the primary electrolysis bath 10 by applying voltage between the anode 11 and the cathode 12 (primary electrolysis step). In this way, the chlorine atoms in the middle chamber 17 pass through the anion-exchange membrane 13 into the anode chamber 15, and these are the chlorine atoms that are converted to chlorine at the anode 11 (Equation (i)). In this way, primary electrolyzed water 6a is produced in the anode chamber 15. Alkaline water 5 is produced in the cathode chamber 16.

Next, alkaline water 3 is added to the primary electrolyzed water 6a produced in the anode chamber 15 to create raw acidic electrolyzed water 6c having an effective chlorine concentration of 10 ppm or more, and containing metal ions at a predetermined concentration (a metal ion concentration (molar equivalent ratio) of from 1.23 to 2.54 relative to the effective chlorine concentration), and this raw acidic electrolyzed water 6c is electrolyzed (second electrolysis step).

This electrolysis yields a secondary electrolyzed water 7 (the acidic electrolyzed water of the present embodiment) having an effective chlorine concentration of 10 ppm or more, and containing metal ions at a predetermined concentration (a metal ion concentration (molar equivalent ratio) of from 0.46 to 1.95 relative to the effective chlorine concentration).

In the acidic electrolyzed water manufacturing device 100A in FIG. 2 (A), primary electrolyzed water 6a having a high degree of purity can be produced in the primary electrolysis bath 10. The acidic electrolyzed water manufacturing device 100A can be readily created by using a commercially available electrolyzed water manufacturing device as the primary electrolysis bath 10, and attaching another electrolyzed water manufacturing device in the rear to serve as the secondary electrolysis bath 20.

The acidic electrolyzed water manufacturing device 100B shown in FIG. 2 (B) has the same configuration and functions as the acidic electrolyzed water manufacturing device 100A shown in FIG. 2 (A) except that, instead of producing raw acidic electrolyzed water 7 by adding alkaline water 3 to the primary electrolyzed water 6a as in the acidic electrolyzed water manufacturing device 100A shown in FIG. 2 (A), alkaline water 4 containing metal ions of an alkali metal or alkaline-earth metal are added to the raw water 1 before the raw water 1 is introduced to the anode chamber 15, and the raw water 1 containing the metal ions is introduced to the anode chamber 15, and the primary electrolyzed water 6b containing metal ions produced in the anode chamber 15 is introduced to the secondary electrolysis bath 20.

In other words, acidic electrolyzed water manufacturing device 100B, as shown in FIG. 2 (B), includes a means for adding metal ions to the raw water 1 before the raw water 1 containing metal ions is introduced to the anode chamber 15.

Here, the metal ions can be added to the raw water 1 in the form of alkaline water 4 containing the metal ions. The alkaline water 4 is preferably an aqueous solution containing cations (metal ions) of an alkali metal or alkaline-earth metal.

More specifically, in the primary electrolysis bath 10 of the acidic electrolyzed water manufacturing device 100B shown in FIG. 2 (B), raw water 1 including alkaline water 4 containing cations (metal ions) of an alkali metal or an alkaline-earth metal is introduced to the anode chamber 15, a chlorine-based electrolyte aqueous solution is introduced to the middle chamber 17, and raw water 2 is introduced to the cathode chamber 16, and the primary electrolyzed water 6b is obtained in the anode chamber 15 (the primary electrolysis step).

In other words, primary electrolyzed water 6b having an effective chlorine concentration of 10 ppm or more, and containing metal ions at a predetermined concentration (a metal ion concentration (molar equivalent ratio) of from 1.23 to 2.54 relative to the effective chlorine concentration) is obtained in the anode chamber 15.

Next, the primary electrolyzed water 6b (raw acidic electrolyzed water 6c) is introduced to the secondary electrolysis bath 20, and electrolysis is performed (the second electrolysis step) to obtain secondary electrolyzed water (the acidic electrolyzed water of the present embodiment) 7.

In the acidic electrolyzed water manufacturing device 100B shown in FIG. 2 (B), the metal ions function as an electrolysis aid when electrolysis is performed on the raw water 1 containing cations (metal ions) of an alkali metal or alkaline-earth metal in the primary electrolysis bath 10. This improves the effectiveness of the electrolysis.

The acidic electrolyzed water manufacturing device 100C shown in FIG. 2 (C) has the same configuration and functions as the acidic electrolyzed water manufacturing device 100A shown in FIG. 2 (A) except that, instead of producing acidic electrolyzed water 7 by adding alkaline water 3 to the primary electrolyzed water 6a as in the acidic electrolyzed water manufacturing device 100A shown in FIG. 2 (A), the alkaline water 5 produced in the cathode chamber 16 is added to the raw water 1 before the raw water 1 is introduced to the anode chamber 15.

In other words, acidic electrolyzed water manufacturing device 100C, as shown in FIG. 2 (C), includes a means for adding alkaline water 5 containing alkali metal ions (sodium ions) generated in the cathode chamber 16 (adding device 44) to the raw water 1 before the raw water 1 is introduced to the anode chamber 15. The alkaline water 5 is produced in the cathode chamber 16 by electrolysis. This alkaline water 5 contains sodium ions (alkali metal ions or cations) derived from the sodium chloride in the chlorine-based electrolyte aqueous solution 9 introduced to the middle chamber 17 of the primary electrolysis bath 10.

More specifically, in the acidic electrolyzed water manufacturing device 100C shown in FIG. 2 (C), electrolysis is performed on the raw water 1 containing sodium ions derived from alkaline water 5 in the primary electrolysis bath 10, and primary electrolyzed water 6b having an effective chlorine concentration of 10 ppm or more, and containing metal ions at a predetermined concentration (a metal ion concentration (molar equivalent ratio) of from 1.23 to 2.54 relative to the effective chlorine concentration) is obtained in the anode chamber 15.

Next, the primary electrolyzed water 6b (raw acidic electrolyzed water 6c) is introduced to the secondary electrolysis bath 20, and electrolysis is performed (the second electrolysis step) to obtain secondary electrolyzed water (the acidic electrolyzed water of the present embodiment) 7.

In the acidic electrolyzed water manufacturing device 100C shown in FIG. 2 (C), the raw water 1 introduced to the cathode chamber 15 contains metal ions (sodium ions) from the alkaline water 5 produced in the cathode chamber 16 of the primary electrolysis bath 10 when electrolysis was performed in the primary electrolysis bath 10, and the metal ions function as an electrolysis aid. This improves the effectiveness of the electrolysis.

In the acidic electrolyzed water manufacturing device 100C shown in FIG. 2 (C), the alkaline water 5 produced in the cathode chamber 16 during electrolysis performed in the primary electrolysis bath 10 can be used to adjust the pH of the raw acidic electrolyzed water 6c and the concentration of metal ions (sodium ions) contained in the raw acidic electrolyzed water 6c electrolyzed in the secondary electrolysis bath 20. As a result, no external additives are required.

The acidic electrolyzed water manufacturing device 100D shown in FIG. 2 (D) has the same configuration and functions as the acidic electrolyzed water manufacturing device 100A shown in FIG. 2 (A) except that, instead of producing raw acidic electrolyzed water 7 by adding alkaline water 3 to the primary electrolyzed water 6a as in the acidic electrolyzed water manufacturing device 100A shown in FIG. 2 (A), the alkaline water 5 produced in the cathode chamber 16 is added to the primary electrolyzed water 6a produced in the anode chamber 15 of the primary electrolysis bath 10, the resulting raw acidic electrolyzed water 6c is introduced to the secondary electrolysis bath 20, and the raw acidic electrolyzed water 6c is electrolyzed (second electrolysis step).

In other words, acidic electrolyzed water manufacturing device 100D, as shown in FIG. 2 (D), includes a means for adding the alkaline water 5 generated in the cathode chamber 16 of the primary electrolysis bath 10 to the primary electrolyzed water 6a produced in the primary electrolysis bath 10.

Here, alkaline water 5 is added to the primary electrolyzed water 6a to obtain raw acidic electrolyzed water 6c having an effective chlorine concentration of 10 ppm or more, and containing metal ions at a predetermined concentration (a metal ion concentration (molar equivalent ratio) of from 1.23 to 2.54 relative to the effective chlorine concentration).

More specifically, in the electrolysis (first electrolysis step) performed in the primary electrolysis bath 10 in the acidic electrolyzed water manufacturing device 100D in FIG. 2 (D), primary electrolyzed water 6a is produced in the anode chamber 15, and alkali water 5 is produced in the cathode chamber 16. Next, the alkaline water 5 is added to the primary electrolyzed water 6a to obtain raw acidic electrolyzed water 6c. The raw acidic electrolyzed water 6c is then introduced to the secondary electrolysis bath 20 and electrolysis is performed to obtain secondary electrolyzed water (the acidic electrolyzed water of the present embodiment) 7.

In the acidic electrolyzed water manufacturing device 100D in FIG. 2 (D), as in the acidic electrolyzed water manufacturing device 100A in FIG. 2 (A), primary electrolyzed water 6a having a high degree of purity can be produced in the primary electrolysis bath 10. The acidic electrolyzed water manufacturing device 100D can also be readily created by using a commercially available electrolyzed water manufacturing device as the primary electrolysis bath 10, and attaching another electrolyzed water manufacturing device in the rear to serve as the secondary electrolysis bath 20.

In the acidic electrolyzed water manufacturing device 100D in FIG. 2 (D), the alkaline water 5 produced in the cathode chamber 16 during electrolysis performed in the primary electrolysis bath 10 can be used to adjust the pH of the raw acidic electrolyzed water 6c and concentration of metal ions (sodium ions) contained in the raw acidic electrolyzed water 6c electrolyzed in the secondary electrolysis bath 20. As a result, no external additives are required.

The following is a more detailed explanation of the present invention with reference to examples. The present invention is not limited to these examples. In the present invention, unless otherwise indicated, “parts” refer to “parts by weight”, and “%” refers to “mass %”.

First, the primary electrolyzed water used in the example was prepared. The primary electrolyzed water was produced using a three-bath electrolyzed water manufacturing device. This electrolyzed water manufacturing device corresponds to the primary electrolysis bath 10 in an acidic electrolyzed water manufacturing device shown in FIG. 2 (A), FIG. 2 (B), FIG. 2 (C), and FIG. 2 (D). When preparing the primary electrolyzed water, sodium chloride was used as the chlorine-based electrolyte. The primary electrolyzed water had an effective chlorine concentration of 100 ppm, a pH value of 2.09, and a sodium concentration of 1 ppm.

In this example, the pH value was measured using a pH measuring device (Handy Digital pH Meter SK-620 PH from Sato Keiryoki Mfg. Co., Ltd.), and the effective chlorine concentration was measured using a chlorine concentration measuring device (Aquab from Shibata Kagaku Co., Ltd.).

Next, raw water and sodium hydroxide were added to 500 ml of primary electrolyzed water obtained in Example 1 to adjust the volume to 1,000 ml. Aqueous solutions having a sodium ion concentration in the primary electrolyzed water of 10 ppm, 20 ppm, 30 ppm, and 40 ppm (primary electrolyzed water) (that is, metal ion (sodium ion) molar equivalent ratio concentrations of 0.62, 1.23, 1.85, and 2.47 (molar equivalent ratio) relative to the effective chlorine concentration) was electrolyzed by applying a 1 A current to an indium oxide anode and a platinum cathode.

The electrolysis in the present example corresponds to the electrolysis performed in the secondary electrolysis bath 20 in the acidic electrolyzed water manufacturing device in FIG. 2 (A). The effective chlorine concentrations in the secondary electrolyzed water prepared in this example (after 60 minutes of electrolysis (sodium ion concentrations: 10 ppm, 20 ppm, 30 ppm, and 40 ppm) was 100 ppm, 134 ppm, 152 ppm, and 160 ppm, respectively. The molar equivalent ratio concentration of metal ions (sodium ions) relative to the effective chlorine concentration was 0.31, 046, 0.61, and 0.77, respectively.

FIG. 4 is a graph showing the relationship between the effective chlorine concentration and the electrolysis time for the acidic electrolyzed water obtained in Example 2. It is clear from FIG. 4 that the effective chlorine concentration rises gradually over time when the sodium hydroxide is added during electrolysis and the sodium ion concentration is 10 ppm.

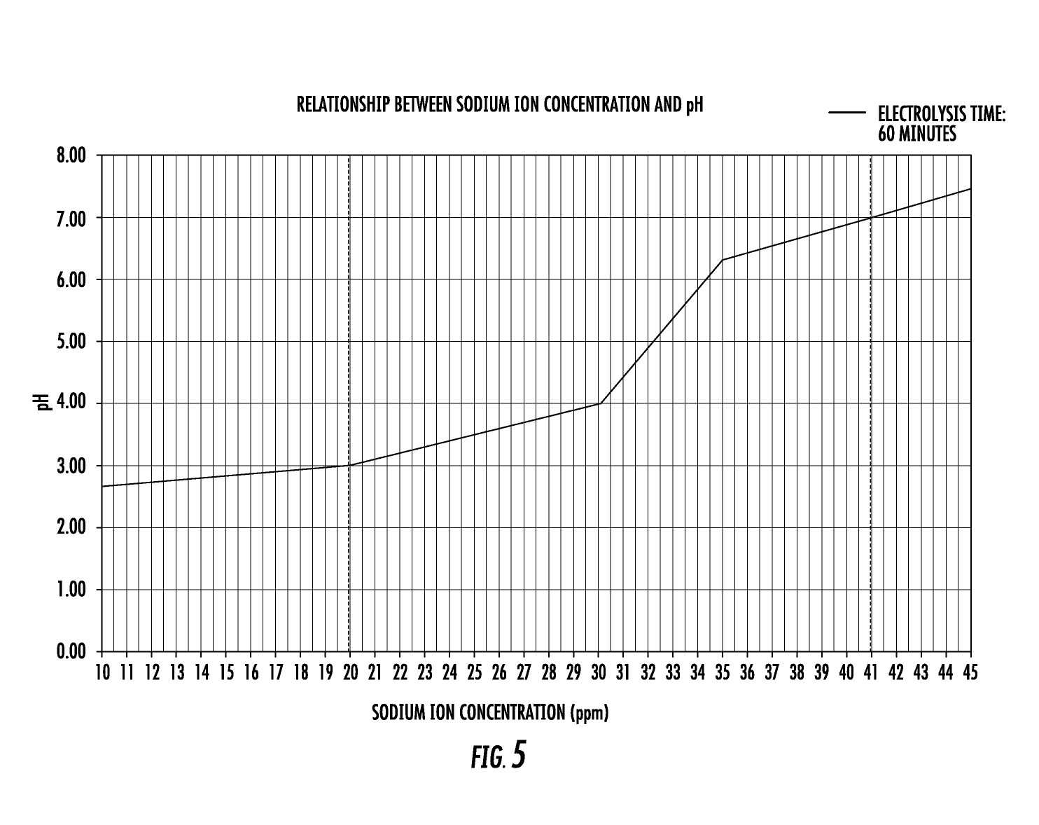

FIG. 5 is a graph showing the relationship between the sodium concentration and the pH of the acidic electrolyzed water (secondary electrolyzed water) obtained in Example 2. It is clear from FIG. 5 that, when electrolysis is performed on raw acidic electrolyzed water containing sodium hydroxide, the effective chlorine concentration is 50 ppm, and the pH of the acidic electrolyzed water is from 3.0 to 7.0 if the sodium ion concentration of the primary electrolyzed water is from 20 ppm to 41 ppm (that is, if the sodium ion molar equivalent concentration relative to the effective chlorine concentration of the raw acidic electrolyzed water is from 1.23 to 2.54).

When raw acidic electrolyzed water having an effective chlorine concentration of 50 ppm was prepared by adding raw water and 0.52 g/L sodium hydroxide to the primary electrolyzed water obtained in Example 1, the raw acidic electrolyzed water was electrolyzed using the same electrodes as Example 2 (applied current: 2 A, electrolysis time: 15 minutes) to prepare a secondary electrolyzed water (sodium concentration: 30 ppm, effective chlorine concentration: 160 ppm). The electrolysis performed in the present example corresponds to the electrolysis performed in the secondary electrolysis bath 20 of the acidic electrolyzed water manufacturing device in FIG. 2 (A).

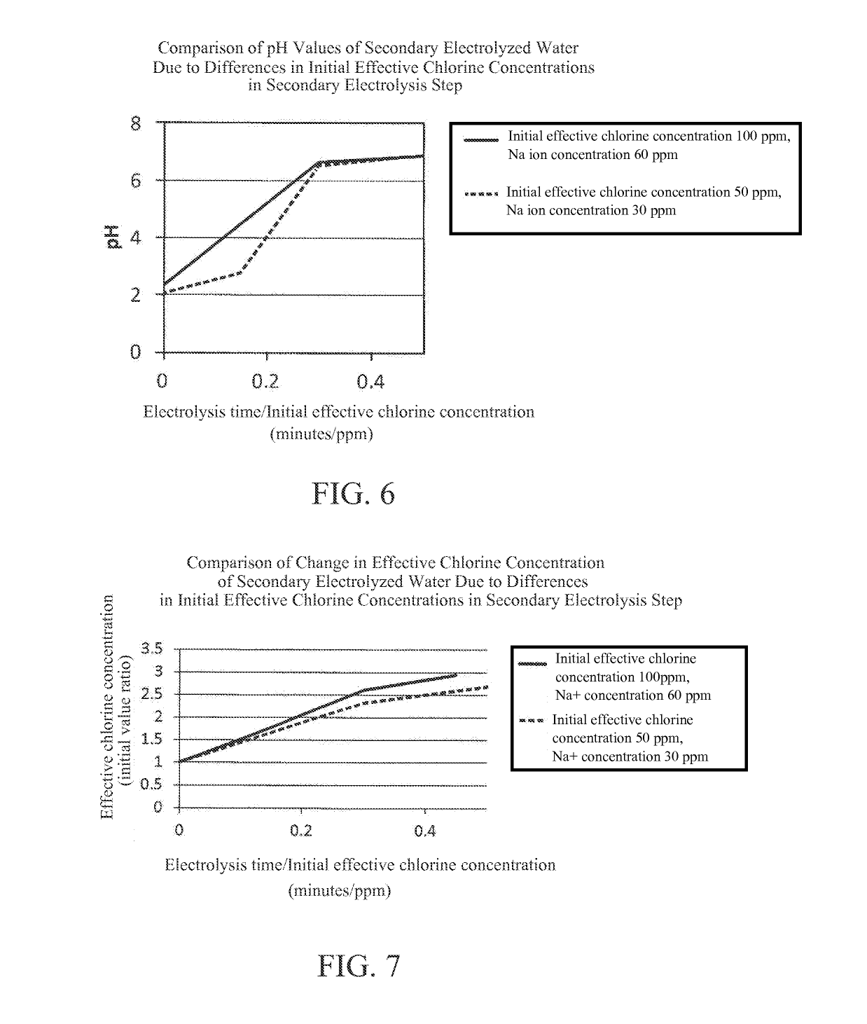

FIG. 6 is a graph showing the relationship between the initial effective chlorine concentration and the pH in the secondary electrolytic step of the present example. FIG. 7 is a graph showing the relationship between the initial effective chlorine concentration and the effective chlorine concentration in the secondary electrolytic step of the present example.

More specifically, raw acidic electrolyzed water 6c in which the effective chlorine concentration was 50 ppm, the sodium ion concentration was 30 ppm (the molar equivalent concentration of sodium ions relative to the effective chlorine concentration was 1.85), and raw acidic electrolyzed water 6c in which the effective chlorine concentration was 100 ppm, the sodium ion concentration was 60 ppm (the molar equivalent concentration of sodium ions relative to the effective chlorine concentration was 1.85) were prepared, and secondary electrolysis was performed (applied current: 2 A).

Because the current applied in the electrolysis was constant, the mass amount reacted per unit of time is not changed by the effective chlorine concentration and sodium ion concentration. Therefore, along the horizontal axis of both FIG. 6 and FIG. 7, the electrolysis time is divided by the initial effective chlorine concentration.

If the percentage of the initial effective chlorine concentration and the sodium ion concentration are the same, the inclination of the pH and effective chlorine concentration are believed to be the same. It is clear from FIG. 6 and FIG. 7 that the pH and effective chlorine concentration change at the same rate if the molar equivalent ratio concentration relative to the effective chlorine concentration is the same even when the concentration of sodium ions is different.

Therefore, acidic electrolyzed water having disinfecting power, acidity (for example, a pH from 3.0 to 7.0), and very little solid content can be obtained by establishing the molar equivalent concentration ratio of the initial effective chlorine concentration and the sodium ions (metal) so that the acidic electrolyzed water of the present embodiment has an effective chlorine concentration of 10 ppm or more, and contains metal ions at a concentration (molar equivalent ratio) from 0.46 to 1.95 relative to the effective chlorine concentration.

When raw acidic electrolyzed water having an effective chlorine concentration of 50 ppm was prepared by adding raw water and 0.052 g/L sodium hydroxide to the primary electrolyzed water obtained in Example 1, the raw acidic electrolyzed water was electrolyzed using the same electrodes as Example 2 (applied current: 2 A, electrolysis time: 15 minutes) to prepare a secondary electrolyzed water (sodium concentration: 30 ppm, effective chlorine concentration: 160 ppm, molar equivalent ratio concentration relative to the effective chlorine concentration: 0.58). The electrolysis performed in the present example corresponds to the electrolysis performed in the secondary electrolysis bath 20 of the acidic electrolyzed water manufacturing device in FIG. 2 (A).

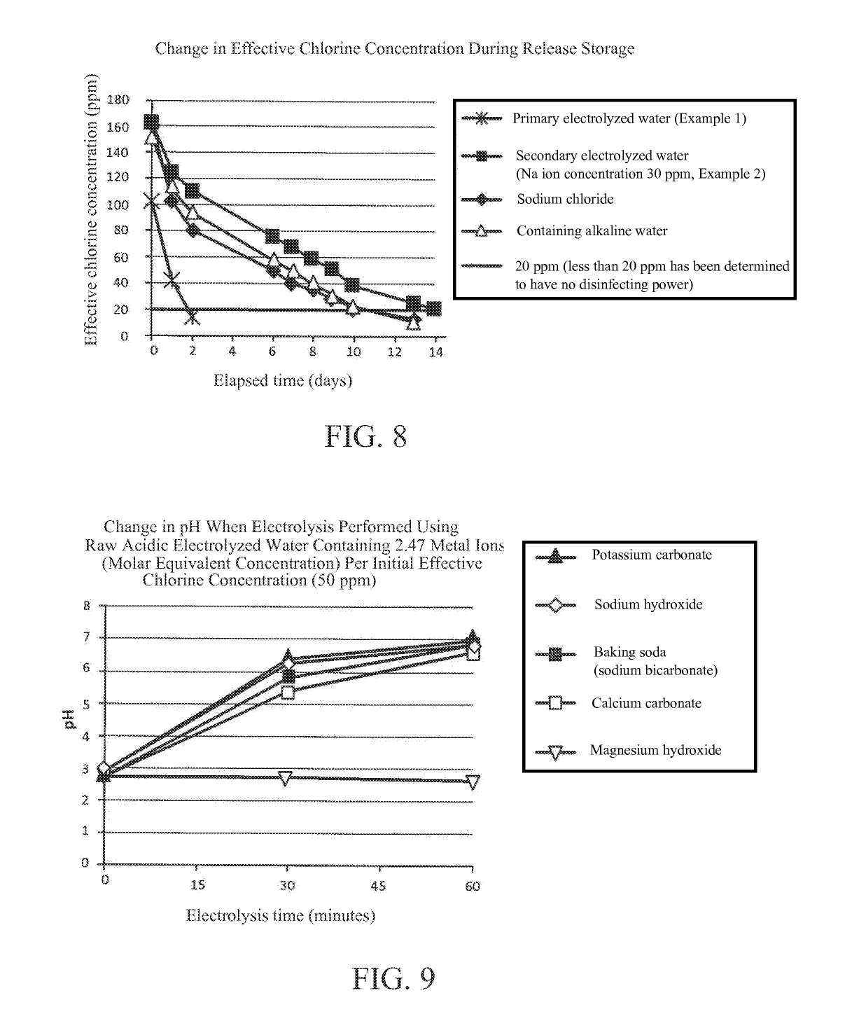

FIG. 8 is a graph showing the change over time in the effective chlorine concentration when the acidic electrolyzed water in the present example was stored openly at room temperature (22° C.). For the sake of comparison, acidic electrolyzed water (sodium ion concentration: 30 ppm) was obtained by adding sodium chloride aqueous solution (sodium chloride concentration: 0.0076 mass %) to the primary electrolyzed water and performing hydrolysis, acidic electrolyzed water (sodium ion concentration: 30 ppm) was obtained by adding alkaline water (pH: 12.64) generated in the cathode chamber 16 to the primary electrolyzed water and performing hydrolysis. These were then were stored openly under the same conditions.

It is clear from FIG. 8 that the acidic electrolyzed water of the present example, which has an initial effective chlorine concentration of 160 ppm and a sodium ion concentration of 30 ppm (metal ion (sodium ion) molar equivalent concentration ratio relative to the initial chlorine concentration: 0.58), had the smallest reduction in effective chlorine concentration and had superior storage stability.

The selected samples were the acidic electrolyzed water obtained by electrolysis in Example 5 (sodium ion concentration: 30 ppm), electrolyzed water obtained by adding a sodium chloride aqueous solution (sodium chloride concentration: 0.0076 mass %) to primary electrolyzed water and performing electrolysis (sodium ion concentration: 30 ppm), acidic electrolyzed water obtained by adding alkaline water (pH: 12.64) produced in the cathode chamber 16 to primary electrolyzed water and performing electrolysis (sodium ion concentration: 30 ppm), and tap water serving as a control. Here, 20 ml of each sample was placed in an open beaker and evaporated into the air over 48 hours at 60° C. and 30% humidity. The mass of the residue remaining in each beaker was measured, and the results are shown in Table 1. In Table 1, the amounts of residue are indicating by the concentration in each liquid.

| | | Acidic | Acidic | |
|---|---|---|---|---|
| | | Electrolyzed | Electrolyzed | |
| | Acidic | Water With | Water With | |
| | Electrolyzed | Added | Added NaCl | |
| | Water in | Alkaline | Aqueous | |
| | Example 5 | Water | Solution | Tap Water |
| Evaporation | 35.80 | 45.93 | 40.75 | 87.24 |
| Residue (ppm) | | | | |

It is clear from Table 1 that the acidic electrolyzed water in Example 5 had less residue than tap water. This is because the amount of residue adhering to internal components of a humidifier (tank, etc.) can be reduced when the acidic electrolyzed water of the present invention is used in the humidifier.

A panel of nine adults confirmed that the acidic electrolyzed water in Example 5 had less odor than tap water (such as the odor of halogens).

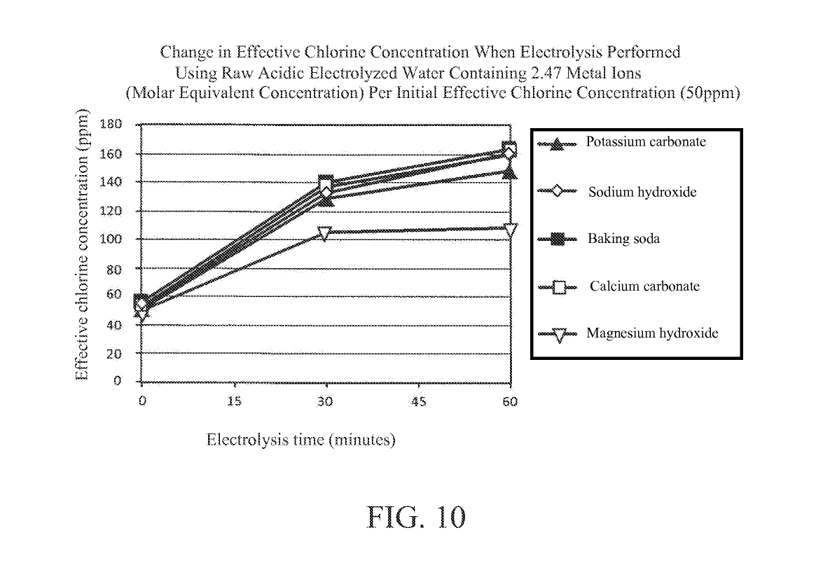

Raw water and electrolytes (potassium carbonate, sodium bicarbonate, calcium carbonate, magnesium hydroxide) including the same equivalent amount of metal ions as the sodium ion concentration (40 ppm, molar equivalent ratio concentration of sodium ions relative to the effective chlorine concentration: 2.47) of the acidic electrolyzed water obtained in Example 2 were added to the primary electrolyzed water obtained in Example 1 (effective chlorine concentration: 100 ppm) to prepare primary electrolyzed water having an effective chlorine concentration of 50 ppm, and then electrolyzing the primary electrolyzed water under the same conditions as Example 2 to obtain secondary electrolyzed water. The results are shown in FIG. 9 and FIG. 10.

FIG. 9 is a graph showing the relationship between the electrolysis time and the pH in each type of acidic electrolyzed water obtained in Example 2 and Example 7. FIG. 10 is a graph showing the relationship between the electrolysis time and the effective chlorine concentration in each type of acidic electrolyzed water obtained in Example 2 and Example 7.

In FIG. 9 and FIG. 10, the acidic electrolyzed water using sodium hydroxide, potassium carbonate, and sodium bicarbonate as the electrolytes had an effective chlorine concentration of 10 ppm or more, contained metal ions at a concentration (molar equivalent ratio) of from 0.46 to 1.95 relative to the effective chlorine concentration, and were acidic (for example, a pH from 3.0 to 7.0). In each example, the change in the effective chlorine concentration during electrolysis was similar. The effective chlorine concentration of the acidic electrolyzed water using magnesium hydroxide as the electrolyte was somewhat lower than the other electrolytes. It is believed that solids adhered to the cathode during electrolysis, which reduced the efficiency of the electrolysis.

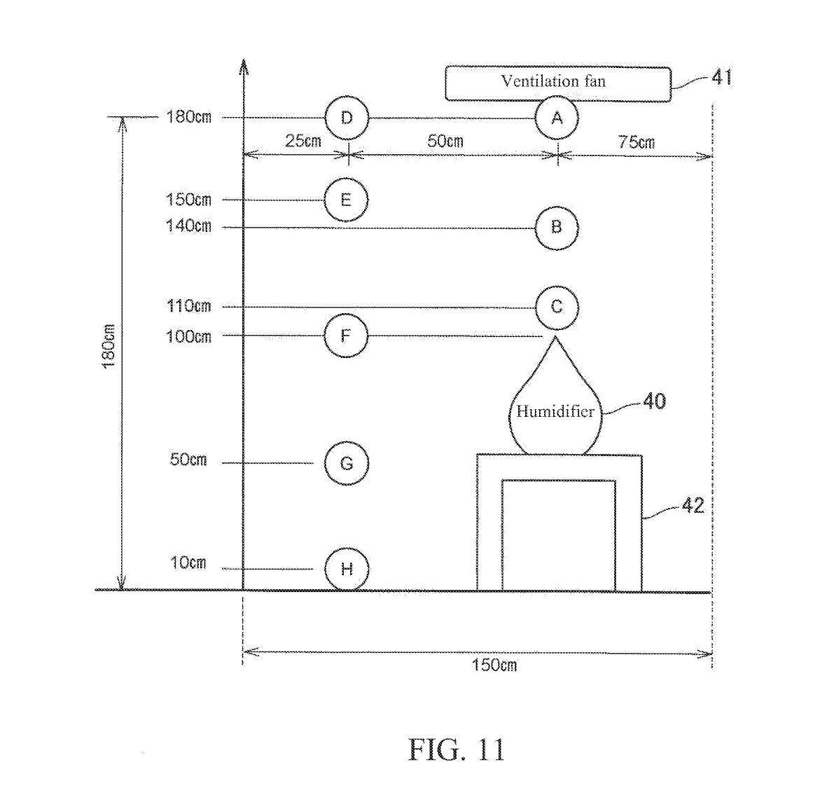

The acidic electrolyzed water obtained in Example 2 was added to the tank of a SHIZUKU AHD-010 ultrasonic aroma humidifier from APIX INTERNATIONAL, the humidifier was operated, and the disinfecting power was evaluated. FIG. 11 is a diagram used to schematically illustrate the method in the disinfecting test conducted on airborne microbes using the acidic electrolyzed water in the present example.

When potassium iodide starch paper was first brought into contact with vapor discharged from the humidifier, the paper turned purple. This confirmed that the vapor discharged from the humidifier contained hypochlorous acid.

Next, a coffee filter was soaked in Candida and then dried for 72 hours at 35° C. and 30% RH to obtain test samples which were placed at positions A, B, C, D, E, F. G and H inside the test booth (150 cm×180 cm×90 cm, W×H×D) shown in FIG. 11. The humidifier 40 was then operated for three hours.

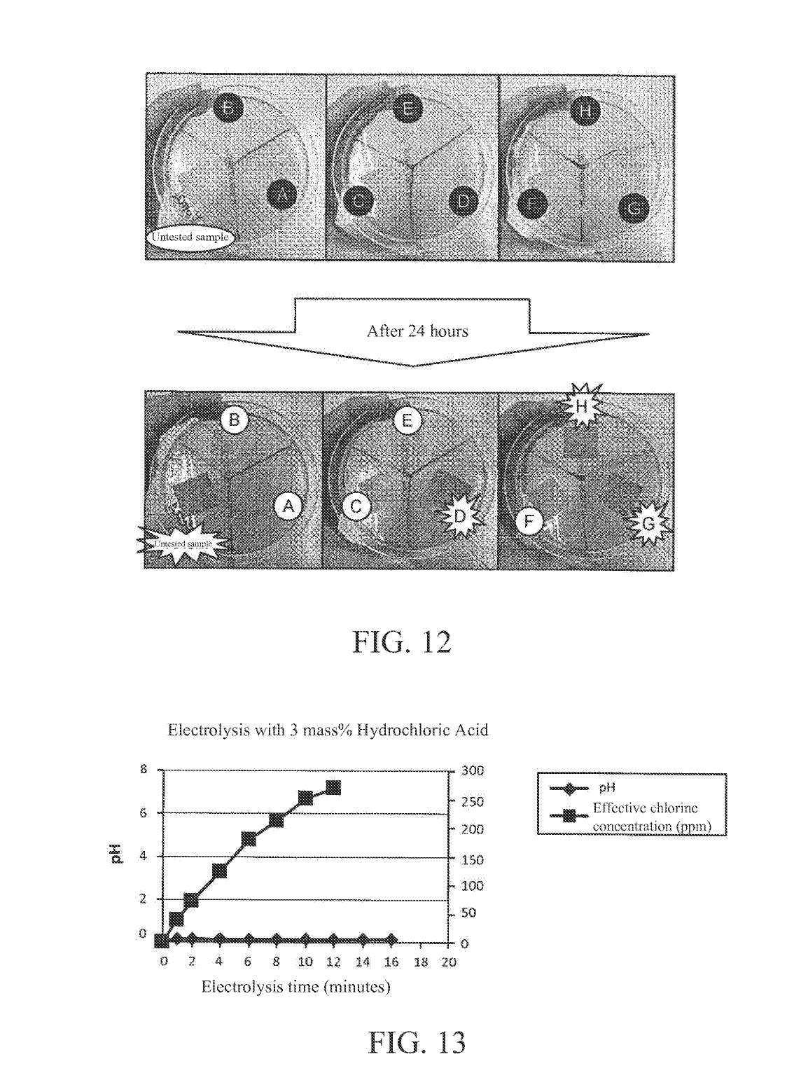

Because the test environment was not hermetically sealed, the test was performed while a ventilation fan 41 with a cleaning filter was operating in order to prevent the spread of microbes to other rooms. After the test, each test sample was allowed to stand for 24 hours in a medium. The results are shown in FIG. 12.

As shown in FIG. 12, while there was no proliferation of the microbe in the test samples at positions A, B, C, E and F, there was proliferation of the microbe in the test samples at positions D, G and H. Because vapor generated by the acidic electrolyzed water of the present invention was present at positions A, B, C, E and F, the microbes in these test samples were killed. The vapor generated by the acidic electrolyzed water of the present invention did not reach positions D, G and H, and so the microbes in these test samples were not killed. Therefore, it is clear that airborne microbes can be killed using acidic electrolyzed water of the present invention.

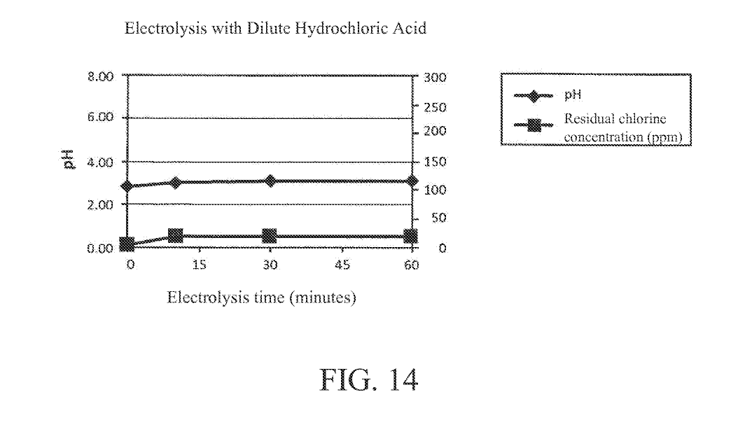

The effect of performing electrolysis with hydrochloric acid at different concentrations is shown in Comparative Examples 1 and 2. The results of performing electrolysis with 3 mass % hydrochloric acid are shown in FIG. 13.

In FIG. 13, electrolysis was performed with 3 mass % hydrochloric acid [in which the molar equivalent concentration ratio of alkali metal ions or alkaline-earth metal ions relative to the effective chlorine concentration is less than 1.23 (nearly zero)]. In this case, the effective chlorine concentration exceeded 300 ppm when the electrolysis time exceeded 14 minutes, and the measuring device could no longer measure the effective chlorine concentration.

In contrast, as shown in FIG. 4, FIG. 9 and FIG. 10, the acidic electrolyzed water in the present invention has both a pH from 3.0 to 7.0, and an effective chlorine concentration of 10 ppm or more.

In Comparative Example 2, electrolysis was performed with an acidic aqueous solution having a pH of 3.0 and a lower hydrochloric acid concentration than the acidic aqueous solution electrolyzed in Comparative Example 1 [in which the molar equivalent concentration ratio of alkali metal ions or alkaline-earth metal ions relative to the effective chlorine concentration is less than 1.23 (nearly zero)]. The results are shown in FIG. 14.

It is clear from FIG. 14 that there was hardly any change over time in the pH value or effective chlorine concentration. It is believed that this occurred because Equation (a) and Equation (iv) in FIG. 1 remained in equilibrium.

From the results shown in FIG. 13 and FIG. 14, it is clear that it is difficult to obtain acidic electrolyzed water of the present invention (electrolyzed water with an effective chlorine concentration of 10 ppm or more and a pH from 3.0 to 7.0) simply by electrolyzing a hydrochloric acid aqueous solution with a low pH or electrolyzing a hydrochloric acid aqueous solution with a high pH.

What is claimed is:

1. Acidic electrolyzed water having an effective chlorine concentration of 10 ppm or more, and containing metal ions at a concentration (molar equivalent ratio) of from 0.46 to 1.95 relative to the effective chlorine concentration, the metal ions being cations of an alkali metal or alkaline-earth metal.

2. Acidic electrolyzed water according to claim 1, wherein the pH value is from 3.0 to 7.0.

3. Acidic electrolyzed water according to claim 1, wherein the solid content is 300 ppm or less.

4. Acidic electrolyzed water according to claim 1, wherein the alkali metal is potassium or sodium.

5. Acidic electrolyzed water according to claim 1, wherein the alkaline-earth metal is calcium or magnesium.

6. A cleanser containing acidic electrolyzed water according to claim 1.

7. A disinfectant containing acidic electrolyzed water according to claim 1.

8. A method for disinfecting microbes contained in air, the method comprising a step of evaporating in the air acidic electrolyzed water according to claim 1.

9. A method for manufacturing acidic electrolyzed water, the method comprising a step of electrolyzing raw acidic electrolyzed water having an effective chlorine concentration of 10 ppm or more, and containing metal ions at a concentration (molar equivalent ratio) of from 1.23 to 2.54 relative to the effective chlorine concentration (where the metal ions are cations of an alkali metal or alkaline-earth metal).

10. A method for manufacturing acidic electrolyzed water according to claim 9, wherein acidic electrolyzed water according to claim 1 is obtained from the step of electrolyzing raw acidic electrolyzed water.

11. A method for manufacturing acidic electrolyzed water according to claim 9 further comprising, prior to the step of electrolyzing raw acidic electrolyzed water, a step of preparing raw acidic electrolyzed water by electrolyzing raw water containing a predetermined concentration of the metal ions and a chlorine-based electrolyte aqueous solution via an anion-exchange membrane.

12. A method for manufacturing acidic electrolyzed water according to claim 9 further comprising, prior to the step of electrolyzing raw acidic electrolyzed water, the steps of:
preparing primary electrolyzed water by electrolyzing raw water and chlorine-based electrolyte aqueous solution via an anion-exchange membrane, and
preparing raw acidic electrolyzed water by adding the metal ions to the primary electrolyzed water.

13. A device for manufacturing acidic electrolyzed water comprising:
a primary electrolysis bath for obtaining raw acidic electrolyzed water by electrolyzing raw water containing a predetermined concentration of metal ions (where the metal ions are cations of an alkali metal or alkaline-earth metal), and
a secondary electrolysis bath for obtaining secondary electrolyzed water by electrolyzing the raw acidic electrolyzed water;
the primary electrolysis bath comprising:
an anode chamber containing an anode,
a cathode chamber containing a cathode, and
a middle chamber provided between the anode chamber and the cathode chamber,
an anion-exchange membrane being provided between the anode chamber and the middle chamber,
a cation-exchange membrane being provided between the cathode chamber and the middle chamber,
the raw water containing metal ions being introduced to the anode chamber,
raw water being introduced to the cathode chamber, and
the chlorine-based electrolyte aqueous solution being introduced to the middle chamber,
the raw acidic electrolyzed water being generated in the anode chamber.

14. A device for manufacturing acidic electrolyzed water according to claim 13, wherein alkaline water containing the metal ions is generated in the cathode chamber, and a means is provided for adding the alkaline water generated in the cathode chamber to raw water prior to the introduction of raw water containing the metal ions to the anode chamber.

15. A device for manufacturing acidic electrolyzed water according to claim 13 further comprising a means for adding the metal ions to raw water provided prior to the introduction of the raw water containing metal ions to the anode chamber.

16. A device for manufacturing acidic electrolyzed water comprising:
a primary electrolysis bath for obtaining primary electrolyzed water by electrolyzing raw water and a chlorine-based electrolyte aqueous solution, and
a secondary electrolysis bath for obtaining secondary electrolyzed water by electrolyzing raw acidic electrolyzed water prepared by adding a predetermined concentration of metal ions (where the metal ions are cations of an alkali metal or alkaline-earth metal) to the primary electrolyzed water;
the primary electrolysis bath comprising:
an anode chamber containing an anode,
a cathode chamber containing a cathode, and
a middle chamber provided between the anode chamber and the cathode chamber,
a cation-exchange membrane being provided between the cathode chamber and the middle chamber,
an anion-exchange membrane being provided between the middle chamber and the anode chamber,
raw water being introduced to the anode chamber and the cathode chamber, and
the chlorine-based electrolyte aqueous solution being introduced to the middle chamber,
the primary electrolyzed water being generated in the anode chamber.

17. A device for manufacturing acidic electrolyzed water according to claim 16 further comprising a means for adding the metal ions to the primary electrolyzed water, raw acidic electrolyzed water being obtained by the means for adding the metal ions.

18. A device for manufacturing acidic electrolyzed water according to claim 16, wherein alkaline water containing metal ions is generated in the cathode chamber, a means is provided for adding the alkaline water to the primary electrolyzed water prior to the introduction of the raw acidic electrolyzed water to the secondary electrolysis bath, and the raw acidic electrolyzed water is obtained by the means for adding the alkaline water.
